(19) United States
(12) Patent Application Publication
(10) Pub. No.: US 2007/0238901 A1
(43) Pub. Date: Oct. 11, 2007

(54) Process for preparing aniline

(76) Inventors: Markus Dugal, Kempen (DE); Franz-Ulrich Von Gehlen, Krefeld (DE); Stefan Wershofen, Monchengladbach (DE); Andre Lago, Shanghai (CN); Peter Lehner, Ratingen (DE); Werner Backer, Wipperfurth (DE); Benie Marotz, Dusseldorf (DE); Horst Brinkschulte, Morsdorf (DE)

Correspondence Address: BAYER MATERIAL SCIENCE LLC, 100 BAYER ROAD, PITTSBURGH, PA, 15205, US

(21) Appl. No.: 11/706,438

(22) Filed: Feb. 15, 2007

(30) Foreign Application Priority Data
Feb. 18, 2006 (DE) 10 2006 007 619.2

(51) Int. Cl. C07C 209/36

(52) U.S. Cl. 564/423

(57) ABSTRACT

Crude aniline is produced by hydrogenating nitrobenzene in the presence of a catalyst. The crude aniline is then extracted with aqueous alkali metal hydroxide solution under conditions such that the aqueous phase is the lower phase during separation of the aqueous and organic phases.

### BACKGROUND OF THE INVENTION

The present invention relates to a process for preparing and purifying aniline by the extraction of crude aniline with aqueous alkali metal hydroxide solution (caustic alkali solution) in which the concentration of the caustic alkali solution used and the temperature are adjusted so that the aqueous phase is the lower phase during phase separation.

Aniline is an important intermediate, e.g., for preparing methylenediphenyl diisocyanate (MDI), and is generally produced on an industrial scale by catalytic hydrogenation of nitrobenzene (See, e.g., DE-OS 2 201 528, DE-OS 3 414 714, U.S. Pat. No. 3,136,818, EP 0 696 573 and EP 0 696 574). In this reaction, in addition to the target product aniline, secondary products such as phenols or aminophenols are also formed and these have to be removed by distillation before further use of the aniline. In particular, the separation of phenol and aniline presents a large challenge to distillation engineering due to their very close boiling points. This difficulty is reflected in the use of long distillation columns with a large number of separating steps and high reflux ratios, with correspondingly high investment and energy costs.

Application JP-A-08-295654 describes, as an alternative to removing phenolic compounds from aniline, an extraction with dilute aqueous caustic soda (or potash) solution in which the majority of the phenol is transferred to the aqueous phase as sodium phenolate. This sodium phenolate is removed as the upper phase by means of subsequent phase separation. Adjusting the concentration of caustic soda solution to <0.7 wt. % is specified as necessary in order to avoid phase inversion and thus problems during phase separation. A molar ratio of NaOH:phenol in the range 3-100:1 is required for effective reduction of the phenol content.

The disadvantage of this disclosed process is the restriction to highly dilute aqueous caustic alkali metal hydroxide solutions of <0.7 wt. % in order to avoid phase separation problems and phase inversion because this means that for a given required molar ratio of alkali:phenol, the amount of alkali metal phenolate-containing effluent may be relatively large. Such large amounts of effluent present ecological and economic disadvantages.

### SUMMARY OF THE INVENTION

The object of the present invention is to provide a simple and economically viable process for the purification of aniline prepared by the catalytic hydrogenation of nitrobenzene in which the costly distillation procedure can be eliminated and at the same time the amount of effluent streams can be reduced.

This object is achieved by extracting crude aniline with an aqueous alkali metal hydroxide solution. The concentration of the aqueous alkali metal hydroxide solution and the temperature at which the extraction is conducted are selected so that the aqueous phase is the lower phase.

### BRIEF DESCRIPTION OF THE DRAWINGS

FIGS. 1 and 2 are schematic representations of preferred embodiments of the process of the present invention.

### DETAILED DESCRIPTION OF THE INVENTION

The present invention is directed to a process for producing aniline by hydrogenating nitrobenzene in the presence of a catalyst to produce crude aniline. The crude aniline is extracted with an aqueous alkali metal hydroxide solution. The aqueous and organic phases formed are separated from each other. The concentration of the alkali metal hydroxide solution used and the temperature during the extraction process are adjusted so that the aqueous phase is the lower phase during separation of the aqueous and organic phases.

The crude aniline may be produced by any conventional industrial process for hydrogenating nitrobenzene. The hydrogenation of nitrobenzene is preferably performed in the gas phase on fixed heterogeneous supported catalysts (such as Pd on aluminum oxide or on carbon supports), in fixed bed reactors at a pressure of 2-50 bar and a temperature in the range of from 250-500° C. under adiabatic conditions using a circulating gas procedure (i.e., with recycling of the unreacted hydrogen from the hydrogenation reaction (See EP-A-0 696 573 and EP-A-0 696 574.)).

It is preferred that the alkali metal hydroxide solution has a concentration >0.7 wt. % of alkali metal hydroxide, with respect to the weight of alkali metal hydroxide solution, in order for the aqueous phase to be the lower phase when separating the aqueous and organic phases. At the same time, however, orientation of the phases may be adjusted by targeted selection of the temperature. The use of higher temperatures, also favors the existence of the aqueous phase as the lower phase, so that even concentrations of alkali metal hydroxide solutions of less than 0.7 wt. % of alkali metal hydroxide, with respect to the weight of alkali metal hydroxide solution, can be used without this leading to renewed re-orientation of the phases.

The process of the present invention also ensures that no phase problems or phase inversions occur during the phase separation procedure included in the extraction process, both when using the preferred alkali metal hydroxide solutions with relatively high concentrations of >0.7 wt. %, with respect to the weight of alkali metal hydroxide solution, and also in the case of optionally used alkali metal hydroxide solutions with low concentrations of less than or equal to 0.7 wt. %, with respect to the weight of alkali metal hydroxide solution. Because the aqueous phase is always the lower phase in the process of the present invention, phase inversion during separation, for example in the phase separation tank, cannot occur.

Sodium hydroxide solution or potassium hydroxide solution is preferably used as the alkali metal hydroxide solution. Sodium hydroxide solution is most preferably used. In principle, however, any alkali metal hydroxide solutions can be used. The use of alkaline earth metal hydroxides or other water-soluble basic compounds such as alkali metal or alkaline earth metal carbonates or hydrogen carbonates is also possible in principle.

The preferred concentration range for the alkali metal hydroxide solution used is between 0.1 and 50 wt. % of alkali metal hydroxide, more preferably between 0.71 and 35 wt. %, most preferably between 0.75 and 10 wt. %, with respect to the weight of alkali metal hydroxide solution.

The temperature of extraction is preferably in the range between 20° C. and 140° C., more preferably between 30° C. and 100° C., most preferably between 50° C. and 95° C., depending on the alkali metal hydroxide concentration. The temperature during the phase separation of the extraction process, is preferably within the same ranges.

The choice of a suitable combination of concentration of alkali metal hydroxide solution and temperature during extraction is governed, in addition to achieving an aqueous phase that is the lower phase during phase separation, by the particular process engineering and economic criteria. Minimization of the temperature may be sensible in order to limit the solubility of aniline in water. It may also be of advantage from a process engineering point of view to condense the crude aniline at an elevated temperature after reaction and then also to extract at that same temperature. Further, too high a concentration of the alkali metal hydroxide solution leads to reduced extraction efficiency and prolonged separation times when, as a result, the organics/water ratio is too high. Too low a concentration of alkali metal hydroxide solution leads to the disadvantage mentioned above of too large an amount of effluent.

The water used to prepare the aqueous alkali metal hydroxide solution is preferably withdrawn entirely or partly from the reaction water from the hydrogenation of nitrobenzene reaction, thereby further reducing the total amount of effluent from the aniline production process. However, water from any other source may also be used. The dilute alkali metal hydroxide solution that is used for extraction is generally produced by adding a concentrated alkali metal hydroxide solution to the feedstock water until the alkali metal hydroxide solution contains the alkali metal hydroxide, e.g. NaOH or KOH, in concentrations which are preferably from 2 to 50 wt. % of alkali metal hydroxide, with respect to the weight of alkali metal hydroxide solution.

Any method and any equipment known to a person skilled in the art, such as mixer-settlers or extraction columns, may be used to conduct the extraction required in the present invention. Extraction may take place in a single step or in several steps, in cocurrent or countercurrent. In a preferred embodiment, a two-step counterstream mixer-settler apparatus is used for the extraction. To shorten the separating and residence times required, the settler may be provided with coalescence aids such as knitted fabrics, plates or packings.

The purified aniline produced by the process of the present invention preferably contains less than 0.01 wt. %, more preferably less than 0.005 wt. % in total of phenolic compounds, with respect to the weight of aniline. In addition to phenol and phenolate, the term phenolic compounds also includes those benzene derivatives which contain other functional groups in addition to the OH function, such as aminophenols.

Other working up steps, such as distillation or washing steps, may be conducted upstream and/or downstream of the extraction with alkali metal hydroxide solutions in order to achieve even higher degrees of purity for the aniline, but are not necessarily required. The downstream or upstream washing and/or distillation steps may be arranged in any variant familiar to a person skilled in the art and may be operated under a wide range of conditions. Thus, distillation may be performed, e.g., in one or more columns with bubble-cap trays or packing, but also in dividing wall distillation columns. Separation of the low-boiling components and high-boiling components may take place in different columns, but also together in one column with side-stream withdrawal of the aniline.

Working up the crude aniline, from which phenolic compounds have largely been removed, by distillation can take place in a variety of ways by adjusting a wide range of conditions. The distillation may be performed in one or several steps in a variety of types of column, preferably in conventional rectifying columns or in those specified as dividing wall distillation columns and with a variety of inserts, such as perforated plates, valve trays or also bubble-cap trays, loose packing or stacked packing. Other embodiments are also possible. The operating parameters, head pressure and reflux ratio always have to be chosen as a function of the composition of the crude aniline, the specification/purity of the purified aniline required (pure aniline) and the separating stages available. The separation of low-boiling components such as water, benzene, cyclohexane, cyclohexylamine, cyclohexanone and higher boiling components such as phenol, alkali metal phenolate, aminophenols, alkali metal aminophenolates, phenylenediamines, diphenylamine etc. may take place separately in different columns or alternatively, in a preferred embodiment, combined in one column with the low-boiling components withdrawn at the head, the high-boiling components withdrawn at the base and the pure aniline withdrawn in a side-stream. Purification of crude aniline, from which phenolic compounds have largely been removed, by distillation takes place in a side-stream column in a preferred embodiment, most preferably in a dividing wall distillation column, with the low-boiling components withdrawn at the head, the high-boiling components withdrawn at the base and pure aniline withdrawn in a side-stream. Further, the base-product from separation of the high-boiling components may optionally be further concentrated in a residuals column in order to minimize the loss of aniline.

The crude aniline from which phenolic compounds have largely been removed may be fed to the distillation column at any position in the column, but introduction preferably takes place in the middle of the column or in the lower half of the column, depending on the concentration profile for aniline in the distillation column. The column may have a stripping and/or strengthening section. The inflow temperature in the column, as well as the base temperature, head pressure and reflux ratio are adjustable and can be adjusted to the separation task as well as to the qualitative, operational and economic requirements. The temperature at the head of the column is set in accordance with the chosen pre-adjustments of the parameters mentioned and the composition of the liquid phase and the vapor phase in the column. Preferred conditions for operating parameters for the distillation column are absolute pressures of 10 to 1000 mbar, most preferably 10 to 500 mbar and reflux ratios of 0.1 to 3, most preferably 0.3 to 0.8.

In a preferred embodiment of the invention, the crude aniline is fed or introduced to a low-boiling column in which the low-boiling components including water are removed via the head of the column. The mixture being produced at the base that contains aniline and high-boiling components is then taken to a further distillation step (removal of high-boiling components or pure distillation). Optionally, concentration of the base mixture from separation of the high-boiling components or pure distillation then takes place on a residuals column. The aniline recovered from the head of the residuals column can be recycled to the column for separation of high-boiling components or pure distillation or to the low-boiling components column or to an upstream phase separation step.

In another preferred embodiment, the crude aniline from which phenolic compounds have largely been removed is fed to a combined low-boiling component and high-boiling component column (side-stream column) in which the low-boiling components are taken away via the head, the high-boiling components are taken from the base and the pure aniline is taken away as a side-stream. This side-stream column can be made up as a conventional column (i.e., without separating partitions) or as a dividing wall distillation column. This variant, in which a side-stream column or a separating partition column is used, requires phase separation of the condensed vapors withdrawn at the head and that substantially contain azeotropic water/aniline and the low-boiling components. Water and low-boiling components dissolved in the aqueous phase are preferably taken away, the aniline is preferably recycled to the column.

The vapors withdrawn at the head of the side-stream column in this embodiment of the process of the present invention are preferably condensed in a two-stage condensation process. The first condenser then preferably partially condenses the higher-boiling components in the vapors. In the second downstream condenser, the low-boiling components that have passed through the first stage are preferably condensed and can thus be removed separately. The partial condensate from the first condenser is taken to a phase separation procedure. Water and the low-boiling components dissolved in the aqueous phase are preferably taken away, the aniline is preferably returned to the column.

Some of the pure aniline withdrawn in the side-stream is preferably fed to the side-stream column as a reflux stream below the withdrawal point of the side-stream. Side-stream withdrawal may be designed as total withdrawal or as partial withdrawal. In both cases targeted adjustment of the reflux ratio can be achieved. The alkali metal hydroxide solution used for extraction can be recycled after the extraction process and used again for extraction purposes, optionally after additional purification and/or concentration. Alternatively, the alkali metal hydroxide solution used for extraction, optionally after additional purification, can be taken to an effluent stream that is taken, for example after subsequent processing, to an effluent treatment plant.

The aniline produced by the process of the present invention may then be reacted with formaldehyde in the presence of an acid catalyst to give di- and polyamines of the diphenylmethane series by any process known to those skilled in the art. These di- and polyamines can then be reacted with phosgene to give the corresponding di- and polyisocyanates in the diphenylmethane series by any process known to those skilled in the art.

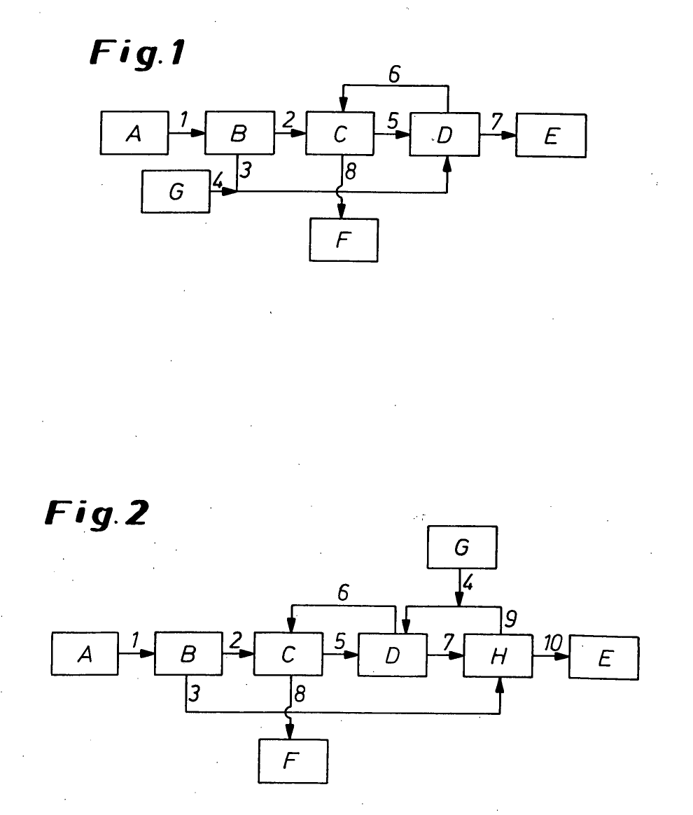

FIG. 1 illustrates a preferred embodiment of the process of the present invention. The mixture 1 of crude aniline and reaction water is transferred from reaction section A, a plant for preparing crude aniline, to a phase separator B. After separating the aqueous phase, the crude aniline 2 is passed to a first mixer-settler extraction step C. The water 3 is adjusted to the desired NaOH concentration by adding caustic soda solution 4 from storage container G and passed to the second mixer-settler extraction step D. The aniline 5 that has been extracted once is passed from the first extraction step C to the second extraction step D, while the aqueous caustic soda solution 6 separated as the lower phase is transferred in counterstream from the second extraction step D to the first extraction step C. The aniline 7 that has been extracted twice is then taken to distillation processing step E, the aqueous alkali solution 8 separated as the lower phase is taken from the first mixer-settler extraction step C to an effluent processing step F.

FIG. 2 shows an alternative, and also preferred, embodiment of the process of the present invention. The mixture 1 of crude aniline and reaction water is transferred from reaction section A, a plant for preparing crude aniline, to a phase separator B. After separating the aqueous phase in phase separator B, the crude aniline 2 is passed to a first mixer-settler extraction step C. The reaction water 3 separated in phase separator B is first taken to a washing step H in which the aniline 7 that has been extracted twice with NaOH solution is washed prior to distillation step E. The water 9 separated from washing step H is treated with caustic soda solution 4 from storage container G and is transferred to the second extraction step D. The aniline 5 that has been extracted once is passed from the first extraction step C to the second extraction step D, while the aqueous caustic soda solution 6, separated as the lower phase, is transferred in counterstream from the second extraction step D to the first extraction step C. The aniline 7 that has been extracted twice is fed as stream 10 to a distillation processing step E, after washing step H. The aqueous alkali solution 8 separated as the lower phase is passed from the first mixer-settler extraction step C to an effluent processing step F.

In another modification of the embodiments described above, the aqueous alkali solution 8 may be circulated and, while optionally removing some of the stream and topping up with fresh alkali solution, again transferred to the second mixer-settler extraction step D for extraction purposes.

Alternatively, the extraction process in both modes of working may also be specified as a single-step or as a more than two-step process.

### EXAMPLES

Examples for performing the process of the present invention are given below. The phenol contents in the following examples were determined by gas chromatographic (GC) analysis. The sodium contents were determined by atomic absorption spectroscopic (AAS) analysis.

### Example 1

A phenol-containing crude aniline is purified by the process of the present invention and purified aniline (pure aniline) was obtained. With a predefined weight ratio of organic phase to aqueous phase of 4.9:1, the phenol present in the crude aniline was depleted with 2.5 wt. % caustic soda solution (2.5 wt. % NaOH with respect to the weight of NaOH solution) using a two-step counterstream extraction process in mixer-settler equipment. The aqueous phase was the lower phase in the phase separation tanks (settlers). The operating parameters are given in Table 1. The phenol is depleted from 939 ppm to 35 ppm (Table 1).

TABLE 1

| | | Amount | | | | | | |
|---|---|---|---|---|---|---|---|---|
| | | of | | | | Phase | | Phenol |
| | Phenol | aniline | | | | ratio in | | in |
| | in crude | phase | NaOH | NaOH | NaOH | parts by | Molar | discharged |
| Temperature | aniline | introduced | solution | solution | conc. | wt. | excess | extract |
| ° C. | ppm | g/h | g/h | ml/h | wt. % | OP/AP | x times | ppm |
| 90 | 939 | 1950 | 400 | 390 | 2.5 | 4.90 | 12.85 | 35 |

(conc. = concentration, OP/AP = organic phase/aqueous phase)

In a subsequent distillation in a side-stream column, pure aniline was withdrawn as the side-stream product. The operating parameters and phenol depletion are reported in Table 2.

TABLE 2

| | | | | | Side- | low/- | | Head | |
|---|---|---|---|---|---|---|---|---|---|
| Feed- | | | Operating | | stream | high- | | condensate | Org. |
| stock | Phenol | Reflux | pressure | Side- | phenol | boiling | Water | aqu. | phase |
| introduced | introduced | ratio | mbar | stream | content | components | conc. | phase | (circulated) |
| kg/h | Ppm | R/E | (abs) | kg/h | ppm | ppm | ppm | g/h | g/h |
| 2.1 | 35 | 1.1 | 133 | 1.8 | 10 | 38/15 | 1000 | 206 | 2.7 |

### Example 2

With a defined weight ratio of organic phase to aqueous phase of 3.87:1, the phenol content present in the crude aniline was depleted from 388 to 26 ppm with 0.8 wt. % caustic soda solution (0.8 wt. % NaOH with respect to the weight of NaOH solution) using a two-step counterstream extraction process in mixer-settler equipment. The operating parameters are reported in Table 3. The aqueous phase was the lower phase in the phase separation tanks (settlers).

TABLE 3

| | Phenol | Amount | | | | Phase | | |
|---|---|---|---|---|---|---|---|---|
| | in | of aniline | | | | ratio in | | Phenol in |
| | crude | phase | NaOH | NaOH | NaOH | parts | Molar | discharged |
| Temperature | aniline | introduced | solution | solution | conc. | by wt. | excess | extract |
| ° C. | ppm | g/h | g/h | ml/h | wt. % | OP/AP | x times | ppm |
| 90 | 388 | 2420 | 624.7 | 620 | 0.8 | 3.87 | 12.52 | 26 |

In a subsequent distillation in a side-stream column, pure aniline was withdrawn as the side-stream product. The operating parameters, the concentrations and phenol depletion achieved are reported in Table 4.

TABLE 4

| | | | | | Side- | low/- | | Head | |
|---|---|---|---|---|---|---|---|---|---|
| Feed- | | | Operating | | stream | high- | | condensate | Org. |
| stock | Phenol | Reflux | pressure | Side- | phenol | boiling | Water | aqu. | phase |
| introduced | introduced | ratio | mbar | stream | content | components | conc. | phase | (circulated) |
| kg/h | ppm | R/E | (abs) | kg/h | ppm | ppm | ppm | g/h | g/h |
| 2.1 | 26 | 0.8 | 133 | 1.8 | 9 | 81/72 | 1100 | 190 | 2.3 |

### Example 3

50 g of a phenol-containing crude aniline were extracted in a two-step cross-stream extraction in separating funnels at 90° C. with a ratio of organic phase to aqueous phase of 5.0:1, using 1.5 wt. % NaOH solution (1.5 wt. % NaOH with respect to the weight of NaOH solution). The aqueous phase in the phase separation tanks (settlers) was the lower phase. The purified aniline obtained was fed to a water-wash procedure to reduce the residual Na content. The phenol content in the crude aniline was thereby reduced from 494 ppm to 50 ppm. As a result of the subsequent water-wash procedure, the phenol content was lowered from 50 ppm to 40 ppm, the Na content in the organic phase dropped from 27 ppm to 9 ppm (See table 5.). The operating parameters are also reported in Table 5.

TABLE 5

| | | Discharge | Discharge | Discharge |
|---|---|---|---|---|
| | Crude | from 1st | from 2nd | from water- |
| | aniline | extraction step | extraction step | wash |
| Phenol (ppm) | 494 | 140 | 50 | 40 |
| Na (ppm) | 0.9 | 72 | 27 | 9.4 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of aniline comprising:
a) hydrogenating nitrobenzene in the presence of a catalyst to produce crude aniline, and
b) extracting the crude aniline with an aqueous alkali metal hydroxide solution to form an aqueous phase and an organic phase, and
c) separating the aqueous and organic phases from each other, in which concentration of the alkali metal hydroxide solution and temperature during the extraction process are adjusted so that the aqueous phase is the lower phase during separation of the aqueous and organic phases.

2. The process of claim 1 in which a) is conducted in gas phase under adiabatic conditions in a fixed bed reactor in the presence of a palladium-containing catalyst.

3. The process of claim 2 in which unreacted hydrogen is recycled.

4. The process of claim 1 in which the alkali metal hydroxide solution is prepared by diluting a more highly concentrated alkali metal hydroxide solution with water.

5. The process of claim 4 in which at least some of the water is produced during a).

6. The process of claim 1 in which sodium and/or potassium hydroxide is used as the alkali metal hydroxide.

7. The process of claim 1 in which the alkali metal hydroxide solution contains the alkali metal hydroxide in a concentration between 0.71 and 35 wt. %, with respect to the weight of the alkali metal hydroxide solution.

8. The process of claim 1 in which the extraction is performed at temperatures of from 20° C. to 140° C.

9. The process of claim 1 in which the alkali metal hydroxide solution used is purified and concentrated after c) and then recycled to b).

10. The process of claim 1 in which the crude aniline prior to b) and/or the purified aniline obtained after c) is purified in a single- or multi-step distillation.

11. The process of claim 1 in which the aniline obtained after c) is purified in a single- or multi-step water-wash procedure.

12. The process of claim 11 in which the purified aniline is further purified in a single- or multi-step distillation.

13. The process of claim 10 in which the distillation is conducted in one step in a side-stream column with low-boiling components being withdrawn at the column head, high-boiling components being withdrawn at the column base and pure aniline being withdrawn as a side-stream.

14. The process of claim 10 in which the distillation is conducted in one step in a dividing wall distillation column with low-boiling components being withdrawn at the column head, high-boiling components being withdrawn at the column base and pure aniline being withdrawn as a side-stream.

15. The process of claim 13 in which vapors withdrawn at the column head are condensed in a two-step condensation process.

16. The process of claim 14 in which vapors withdrawn at the column head are condensed in a two-step condensation process.

17. A process for preparing di- and polyamines in the diphenylmethane series comprising reacting aniline produced by the process of claim 1 with formaldehyde in the presence of an acid catalyst.

18. A process for preparing di- and polyisocyanates in the diphenylmethane series comprising reacting a diamine or polyamine produced by the process of claim 17 with phosgene.
